(19) United States
(12) Patent Application Publication
(10) Pub. No.: US 2007/0292492 A1
(43) Pub. Date: Dec. 20, 2007

(54) PHARMACEUTICAL FORMULATIONS FOR IONTOPHORETIC TETRACYCLINE ANTIBIOTIC DELIVERY

(76) Inventor: Phllip M. Friden, Bedford, MA (US)

Correspondence Address: ELMORE PATENT LAW GROUP, PC, 209 MAIN STREET, N. CHELMSFORD, MA, 01863, US

(21) Appl. No.: 11/762,966

(22) Filed: Jun. 14, 2007

Related U.S. Application Data
(60) Provisional application No. 60/814,609, filed on Jun. 16, 2006.

(51) Int. Cl. A61K 31/65; A61F 13/02; A61N 1/30

(52) U.S. Cl. 424/448; 514/152; 604/20

(57) ABSTRACT

Pharmaceutical formulations suitable for iontophoresis that provide enhanced iontophoretic delivery of tetracycline antibiotic to at least one target tissue are described and methods for administering tetracycline antibiotic via iontophoresis.

### RELATED APPLICATION

This application claims the benefit of U.S. Provisional Application No. 60/814,609, filed on Jun. 16, 2006. The entire teachings of the above application are incorporated herein by reference.

### BACKGROUND OF THE INVENTION

An iontophoretic delivery system is, for example, a drug delivery system that releases drug at a controlled rate to the target tissue upon application. The advantages of systems wherein drug is delivered locally via iontophoresis are the ease of use, being relatively safe, and affording the interruption of the medication by simply peeling off or removing from the skin whenever an overdosing is suspected. The total skin surface area of an adult is about 2 m². In recent years iontophoretic delivery of drugs has attracted wide attention as a better way of administering drugs for local as well as systemic effects. The design of iontophoretic delivery systems can usually be such that the side effects generally seen with the administration of conventional dosage forms are minimized.

Iontophoresis has been employed for many years as a means for applying medication locally through a patient's skin and for delivering medicaments to the eyes and ears. The application of an electric field to the skin is known to greatly enhance the ability of the drugs to penetrate the target tissue. The use of iontophoretic transdermal delivery techniques has obviated the need for hypodermic injection for some medicaments, thereby eliminating the concomitant problems of trauma, pain and risk of infection to the patient.

Iontophoresis involves the application of an electromotive force to drive or repel ions through the dermal layers into a target tissue. Particularly suitable target tissues include those adjacent to the delivery site for localized treatment. Uncharged molecules can also be delivered using iontophoresis via a process called electroosmosis.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the site of drug delivery and a passive or “grounding” electrode affixed to a second site on the skin to enhance the rate of penetration of the medicament into the skin adjacent to the applicator electrode.

U.S. Pat. No. 6,477,410 to Henley et al. describe the use of iontophoresis for drug delivery. However, improved formulations that facilitate the delivery of specific active agents are desired.

### SUMMARY OF THE INVENTION

The present invention provides pharmaceutical formulations suitable for iontophoresis that provide enhanced iontophoretic delivery of tetracycline antibiotic to at least one target tissue. The formulations are further characterized by good to excellent stability. The present invention also provides methods of administering tetracycline in at least one target tissue of and/or treating acne in a patient by iontophoretically delivering a formulation of the invention.

### BRIEF DESCRIPTION OF THE DRAWINGS

FIG. 1 is a graph illustrating the difference in passive [symbol] and anodal [symbol] iontophoresis of tetracycline hydrochloride with a current density of 0.4 mA/cm² applied for 1 hour.

FIG. 2 is a graph showing the difference in passive [symbol] and cathodal [symbol] iontophoresis of tetracycline hydrochloride for 1 hour using 0.4 mA/cm².

FIG. 3 illustrates the cumulative amount of tetracycline permeated for 1 hour with an increase in the current density; [symbol] 0.2 mA/cm², [symbol] 0.3 mA/cm², [symbol] 0.4 mA/cm².

FIG. 4 shows a cumulative amount of tetracycline permeated through the skin with the increase in time of application of current; [symbol] 0.4 mA/cm² for 30 min, [symbol] 0.4 mA/cm² for 1 h, [symbol] 0.4 mA/cm² for 2 h.

FIG. 5 depicts the cumulative amount of tetracycline permeated as a function of increasing donor concentration; donor concentrations: [symbol] 2.5 mg/ml, [symbol] 5 mg/ml, [symbol] 10 mg/ml.

FIG. 6 is a graph showing results from tetracycline microdialysis studies conducted at a drug concentration of 10 mg/mL and at a pH of approximately 9.3.

### DETAILED DESCRIPTION OF THE INVENTION

In one aspect, the invention provides pharmaceutical formulations that are suitable for iontophoresis and that provide enhanced iontophoretic delivery of at least one tetracycline antibiotic to a patient, preferably a human patient, in need of treatment.

Tetracycline antibiotics have been known for treatment of acne, rosacea and perioral dermatitis. Tetracycline antibiotics include, but are not limited to, tetracycline, chlortetracycline, oxytetracycline, demecloycline, doxycycline, lymecyline, meclocyline, methacycline, minocyline, rolitetracycline and tigecycline. Iontophoretic delivery of tetracycline antibiotic can deliver it directly to the diseased skin rather than systemically. Thus, the invention relates to the iontophoretic delivery of tetracycline antibiotic, including cathodal or anodal iontophoresis. In a preferred embodiment, the tetracycline antibiotic is formulated in a buffer at a pH between about 1.5 and 4 (preferably between about 2 and 3.5) or between 7.5 and 11 (preferably between about 7.5 and 9.5). Preferably the ionic strength of the buffer is at least about 0.05M, such as about 0.1 to 0.25M. Preferably, the concentration of the active agent in the formulation is at least about 1 mg/ml, such as at least about 8 mg/ml, preferably at least about 10 to 50 mg/ml, such as between 10 to 25 or 40 mg/ml.

In one preferred embodiment, the tetracycline antibiotic is formulated free of dermal penetration enhancer which is selected from the group consisting of 1-alkylazacyloheptan-2-one, said alkyl having from 8 to 16 carbon atoms, and a cis-olefin of the formula CH₃(CH₂)ₓCH═CH(CH₂)_{y}R³, where R³ is CH₂OH, CH₂NH₂ or COR⁴, and R₄ is OH or (C₁-C₄)alkoxy, x and y are each an integer from 3-13 and the sum of x and y is from 10 to 16.

A formulation of the invention is preferably a viscous formulation. As used herein, the term “viscous formulation” includes colloidal and gel formulations, such as a viscous formulation having a viscosity of greater than about 500 cp at 25 degrees Celsius. A viscosity modifying agent can be added to the formulation to achieve the desired viscosity. The pharmaceutically acceptable carrier or excipient may comprise about 0.1 to 10 weight percent of a viscosity modulating agent. As used herein, the term “pharmaceutically acceptable carrier or excipient” means any non-toxic diluent or other formulation auxiliary that is suitable for use in iontophoresis. Examples of pharmaceutically acceptable carriers or excipients include but are not limited to: diluents such as water, or other solvents, cosolvents; solubilizing agents such as sorbital and glycerin; buffers such as, for example, phosphate buffer solutions; pharmaceutically acceptable bases; and viscosity modulating agents such as cellulose and its derivatives.

The viscosity of the viscous formulation may be controlled by a viscosity modulating agent. A viscosity modulating agent includes any agent that is capable of modulating the viscosity of a gel. Viscosity modulating agents useful in the practice of the invention include but are not limited to, ionic and non-ionic, high viscosity, water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. If a uniform gel is desired, dispersing agents such as alcohol, sorbitol or glycerin can be added, or the gelling agent can be dispersed by trituration, mechanical mixing, or stirring, or combinations thereof. In one embodiment, the viscosity enhancing agent can also provide the base, discussed below.

In one preferred embodiment, the viscosity modulating agent is cellulose that has been modified such as by etherification or esterification. One such etherified cellulose polymer is sold under the trademark Natrosol® (Hercules-Aqualon, Wilmington, Del.).

In one aspect, the invention provides a pharmaceutical formulation suitable for ionotophoresis that may further comprise at least one antioxidant, stabilizer, chelator, preservative, aldehyde scavenger or mixture thereof. Preferably, the excipients should be uncharged so as not to compete with the tetracycline transport.

The term “antioxidant” is intended to mean an agent which inhibits oxidation and thus is used to prevent the deterioration of preparations by the oxidative process. Such compounds include by way of example and without limitation, acetone, sodium bisulfate, ascorbic acid, alpha-tocopherol, ascorbyl palmitate, citric acid, butylated hydroxyanisole, butylated hydroxytoluene, hydrophosphorous acid, monothioglycerol, propyl gallate, sodium ascorbate, sodium citrate, sodium sulfide, sodium sulfite, sodium bisulfite, sodium formaldehyde sulfoxylate, thioglycolic acid, sodium metabisulfite, EDTA (edetate), pentetate and others known to those of ordinary skill in the art.

The term “stabilizer” is intended to mean a compound used to stabilize a therapeutic agent against physical, chemical, or biochemical process that would otherwise reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art.

The term “chelator” as used herein refers to a molecule that binds metal ions, usually by binding to two or more complexing groups within the molecule. Chelators are well known in the art, and include certain proteins and polypeptides, as well as small molecules such as ethylenediaminetetraacetic acid (EDTA), ethylene glycol-bis(β-aminoethyl ether)-N,N,N′,N′-tetraacetic acid (EGTA), nitrilotriacetic acid, oxalate, citric acid, 1,2-diaminocyclohexane-N,N,N′N′-tetracetic acid, 4,5-dihydroxybenzene-1,3-disulfonic acid, pyrocatechol-3,5-disulfonate, salicylic acid, 5-sulfosalicylic acid, xylenol orange, aurintricarboxylic acid, 2,2′-pyridyl ethylene diamine, glycine, 8-hydroxyquinoline-5-sulfonic acid, lactic acid, 1,10-phenanthroline, pyridine, pyridine-2,6-dicarboxylic acid, 8-quinolinol, succinic acid, tartaric acid, thioglycolic acid, 1,1,1-trifluoro-3,2′-thenolyacetone, triethylene tetramine and the like.

The preservatives include antimicrobial agents that kill and/or inhibit the proliferation and/or growth of microbes, particularly bacteria, fungi and yeast. Preservatives can be synthetic compounds, semisynthetic compounds, and naturally produced compounds. Suitable dermatologically absorbable preservatives include erythromycin, bacitracin, zinc bacitracin, polymycin, neomycin, chloramphenicol, tetracycline, sulfacetamide, minocycline, clindamycin, doxycycline, undecylenic acid and salts thereof, propionic acid and salts thereof, caprylic acid and salts thereof, ciprofloxacin, cephlasporins, benzoic acid, ciclopiroxolamine, clotrimazole, econazole nitrate, metronizadole, miconazole nitrate, ketacanazole, oxiconazole, tolnaftate, benzalkonium chloride, parabens, methyl paraben, benzethonium chloride, Neolone 950, sodium benzoate, sodium bisulfite, phenol, alkyl esters of parahydroxybenzoic acid, o-phenylphenol benzoic acid and salts thereof, boric acid and salts thereof, sorbic acid and salts thereof, chlorobutanol, benzyl alcohol, thimerosal, phenylmercuric acetate and nitrate, nitromersol, and cetylpyridinium chloride.

The term “aldehyde scavenger” as used herein is a substance that reacts with an aldehyde to form a neutralized aldehyde that has decreased ability to form adducts with the amino groups of tetracycline and that does not itself react with tetracycline. Aldehyde scavengers include, for example, substances that contain primary amine groups that react with aldehyde functional group(s). Aldehyde scavengers also include sulfites. Suitable aldehyde scavengers include, but are not limited to, urea, methionine and methionamide.

The term “base” is used in its traditional sense, i.e., a substance that disassociates in water to produce hydroxide ions. Any base may be used provided that the compound provides free hydroxide ions in the presence of water. Such bases include inorganic or organic pharmaceutically acceptable bases. Preferred inorganic bases include inorganic hydroxides, such as alkali metal hydroxides, carbonates, inorganic oxides, inorganic salts of weak acids and combinations thereof. Preferred organic bases are nitrogenous bases, such as amines and quaternary ammonium bases. In one preferred embodiment, the base is NaOH.

The term “buffer” refers to solutions of compounds that are known to be safe for pharmaceutical or veterinary use in formulations and that have the effect of maintaining or controlling the pH of the formulation in the pH range desired for the formulation. Acceptable buffers for controlling pH at a moderately acidic pH to a moderately basic pH include, but are not limited to, such compounds as phosphate, acetate, citrate, borate, arginine, TRIS, and histidine. “TRIS” refers to 2-amino-2-hydroxymethyl-1,3,-propanediol, and to any pharmacologically acceptable salt thereof. Preferable buffers are phosphate or borate buffers with saline or an acceptable salt.

Additionally, a surfactant or wetting agent can be added to facilitate application or wetting of the formulation to the iontophoresis pad material, or drug cartridge pad. Examples of suitable surfactants or wetting agents include surfactants such as polyoxyethylene hydrogenated castor oil 60, polyoxyethylenesorbitan monooleate, polyoxyethylenesorbitan monolaurate, polyoxyethylenelauryl ether, polyoxyethyleneoctyl phenyl ether, polyoxyethylenenonyl phenyl ether, polyoxyethylene polyoxypropylene glycol, polysorbate and saccharose aliphatic acid ester; saccharides such as glucose, maltose, fructose, galactose, mannitol, sorbitol, mannose, glucosamine, lactose, sucrose and trehalose; water-soluble cyclodextrins including natural cyclodextrins such as α-cyclodextrin, β-cyclodextrin and γ-cyclodextrin, water-soluble cyclodextrin derivatives having a substituent including hydroxypropyl, glycolyl, maltosyl, sulfate, phosphate, carboxyl, carboxymethyl, carboxymethylethyl and/or amino, and cyclodextrin polymers; water-soluble polymers such as starches, dextran, dextran sulfate, inulin and polyvinylpyrrolidone; and wetting agents such as glycerol, ethyleneglycol, polyethyleneglycol, propyleneglycol, butyleneglycol, urea, ethylurea, urea derivatives, methylpyrrolidone and pyrrolidone derivatives, may be exemplified.

In one embodiment, formulations of the present invention can be reconstituted prior to iontophoretic delivery. In a preferred embodiment, formulations of the invention can be reconstituted by wicking the excipient(s) layer of the cartridge pad with the active tetracycline antibiotic formulation(s) of the invention before administering iontophoretically.

The present invention further provides a kit for use in iontophoretic delivery of a pharmaceutically acceptable formulation of tetracycline antibiotic, the kit comprising: a glass vial or a MDPE blow fill seal ampoule containing a pharmaceutically acceptable formulation comprising tetracycline antibiotic; and a cartridge or a patch comprising a pharmaceutically acceptable excipient(s) selecting from diluent(s), solubilizing agent(s), preservative(s), viscosity modulating agent(s), buffer system, penetration enhancer(s), stabilizer(s), antioxidant(s), chelator(s) and mixture thereof.

In one preferred embodiment, the kit comprising: a glass vial or a MDPE blow fill seal ampoule containing a pharmaceutically acceptable formulation comprising tetracycline antibiotic in a solution of thioglycerol, propylene glycol or polyethylene glycol, wherein the formulation does not contain a water phase; and a cartridge or a patch comprising a pharmaceutically acceptable excipient(s) selected from diluent(s), solubilizing agent(s), preservative(s), viscosity modulating agent(s), buffer system, penetration enhancer(s), stabilizer(s), antioxidant(s), chelator(s) and mixture thereof.

As used herein the term “target tissue” includes the patient's dermis, epidermis, nails, mucocutaneous membranes including, but not limited to, the eye and the body cavity and canal sites such as mouth, ear, nose, vagina, and rectum.

Preferred iontophoretic delivery devices useful with the compositions and methods of the invention include but are not limited to those described in U.S. Pat. Nos. 6,148,231, 6,385,487, 6,477,410, 6,553,253, and U.S. Patent Publication Numbers 2004/0111051, 2003/0199808, 2004/0039328, 2002/0161324, and U.S. Application Ser. No. 60/743,528, all incorporated herein by reference. A preferred applicator which has been developed for use with a device for electrokinetically delivering a medicament to a treatment site comprising an applicator head having opposite faces and including an active electrode and a porous pad (such as a woven or non-woven polymer, for example, a polypropylene, pad); a margin of the applicator head about the active electrode having a plurality of spaced projections therealong; the porous pad and the applicator head being ultrasonically welded to one another about the margin of the head with the electrode underlying the porous pad; and a medicament or a medicament and an electrically conductive carrier therefor carried by the porous pad in electrical contact with the electrode. Alternatively or additionally, the applicator has been developed for use with a device for electrokinetically delivering a medicament to a treatment site comprising an applicator head having opposite faces and including an active electrode and a porous pad overlying the active electrode; a medicament or a medicament and an electrically conductive carrier therefor carried by the pad and in electrical contact with the electrode; a lid overlying the porous pad on a side of the porous pad remote from the electrode and releasably secured to the applicator head; and the lid comprising layers of different materials and including one or more tabs, one of the layers of the lid and the tab being formed of a metallic material, at least a portion of an interface between the metallic material of the tab and the metallic material of the lid having a discontinuity. In another embodiment, the lid may be an oversided disc having a rim constituting an annular tab. Additionally or alternatively, the applicator which has been developed for use with a device for electrokinetically delivering a medicament to a treatment site comprising an applicator head having opposite first and second faces and including an active electrode and a porous pad overlying said electrode; a medicament or a medicament and an electrically conductive carrier therefor carried by the pad; a margin of the cartridge about the active electrode and a margin of the porous pad being secured to one another; the active electrode having a first portion thereof exposed through the first face of the applicator head remote from the porous pad; and another portion of the active electrode being exposed to the porous pad along the second face of the applicator head for electrical contact with the medicament or the medicament and the electrically conductive carrier.

The formulations are preferably administered via iontophoresis. In a preferred embodiment, a current density of at least 0.02 mA/cm² is applied, such as at least 0.2 mA/cm². In a preferred embodiment, a flux of at least about 1 μg/cm²-hr, such as about 20 μg/cm²-hr, is achieved. The iontophoresis can be applied for a sufficient time to achieve an effective amount of drug in the skin. In general, the time of application can be between about 5 and 60 minutes, such as about 30 minutes or less.

In a further embodiment, micro-needles may advantageously be formed on an outermost, biological interface contacting surface of the iontophoresis device. One such preferred micro-needles device is disclosed in U.S. Publication No. 20070066934; which is incorporated herein by reference in its entirety.

To penetrate one or more high electrically resistant layers to supply medicament to a targeted underlying layer or layers, a pad or applicator is provided having a surface array of needles, preferably micro-needles along one side or face of the applicator. The needles are carried by a non-conductive membrane of the applicator and project from the membrane a distance sufficient to penetrate the high electrically resistant layer(s), upon application of the applicator to the individual's skin. Because of the very high density of the needles, preferably micro-needles, numerous low electrically resistant areas are created by perforating the high electrically resistant layer(s). That is, the needles form a multiplicity of channels i.e., micro-channels through the more highly electrically resistant layer(s). The needles in effect create channels in the skin. The length and density of the needles as well as the thickness or diameter of the needles including the diameter of the orifices through the needles can be varied depending upon the location of the targeted treatment site underlying the skin surface. The needles may be formed of a non-conductive material, e.g., a plastic material or may be formed of metal material coated with a non-conductive material. The needles can be monolithic with well-defined orifices for delivery of actives or fused particulates (sintered) that provide a porous needle with a tortuous network of many liquid transport paths in a more tortuous design. Such sintered material avoids the problem of needle coring of stratum-corneum tissue that occludes the fluid passages. It is understood that such material would include filaments, particles, staple fibers, wires or other forms of needle material that is joined under pressure to create a porous needle structure. Needles may also be made of conductive materials and coated with nonconductive layers. The needles may also be made of non-conductive intermetallic glasses. The needles may also be formed of bioresorbable polymers containing drugs or other active ingredients molecularly dissolved or dispersed as a separate phase. The active ingredient is delivered to the skin electrokinetically as the needle polymer is eroded and/or solubilized by interstitial fluid within the skin. Polymers such as polylactic acid, polyglycolic acid, copolymers of poly(lactide-glycolide), polyorthoesters, polyvinylalcohol and others, as well as natural products such as sugars, starches and graft copolymers of these. The opposite side of the pad from the needles may comprise a conductive membrane in contact with an active electrode and a power supply.

The micro-needles may be attached to a flexible substrate to provide a compliant system for skin interface. Micro-needles may not penetrate the epidermis to the full extent of needle height due to the compliant nature of the stratum-corneum and dermal underlayers. Additionally, skin is a viscoelastomer that relaxes mechanically under load. This causes the substrate to move away from the needle during puncture. One means for improving the consistency of puncture by needle arrays is to impose an upward movement of the skin using an iontophoretic patch. The patch may include a rigid boundary surrounding an array of micro-needles enabling, upon application, the skin surrounded by the boundary to present itself, i.e., become proud of skin adjacent the patch, to the micro-needle array. In another embodiment, to provide skin penetration, the arrays of micro-needles are attached to a slightly concave-shaped elastomeric backing attached to the iontophoretic patch and acts as a suction cup. Upon actuation by the user, the target skin area is pilled into the concavity and against the micro-needles attached to the more rigid backing material. Micro-needles are thus allowed to penetrate the skin without interference from the more compliant dermal layers below. The system also includes a device containing the active and ground electrodes and a power supply. Preferably, the applicator and the device are separable from one another whereby the applicator is disposable and the device may be reused with a fresh applicator. Alternatively, the device and applicator may constitute an integrated disposable or reusable unit.

In one embodiment the micro-needles may be solid such that medicament does not pass through conduits in the needles. The micro-needles may be formed of maltose or other materials that will rapidly dissolve upon contacting fluid within the skin. In this embodiment, the needles are used to perforate the skin and may or may not be used to apply medicament. A least a portion of the needles dissolved in the skin. The dissolving of the needles may be simultaneous with the application of current for electrophoreses to drive the medicament to the treatment site is at or underlines the pores created by the micro-needles. Alternatively, the dissolving needles may not be embedded in a medicament pad of the applicator. The solid micro-needles skin perforate the skin to form pores in the skin, such as through the stratum corneum. The needles dissolve or be otherwise removed from the pores. Thereafter, the electrokinetic applicator infuses medicament from the medicament pad, through the pores formed by the needles and into the treatment site underlying the skin surface. By establishing an electrical current through the active electrode, medicament pad and skin, the medicament, e.g. tetracycline antibiotic, is delivered through pores created by the needles and into the skin, e.g., the epidermis, by iontophoresis.

The system also includes a device containing the active and ground electrodes and a power supply. Preferably, the applicator and the device are separable from one another whereby the applicator is disposable and the device may be reused with a fresh applicator. Alternatively, the device and applicator may constitute an integrated disposable or reusable unit.

In one embodiment, the applicator containing the needles may be combined with a delivery device. For example, the finger mounted devices disclosed in U.S. Pat. Nos. 6,792,306 and 6,735,470, may be provided with applicators containing needles of selected sizes and configurations to penetrate through the high electrically resistant layers of the skin to supply medicament to the targeted treatment site. Alternatively, the device disclosed in U.S. Pat. No. RE37796, may likewise use applicators of the type described herein. In all instances, by forming a multiplicity of low electrically resistant perforations or pathways through the higher electrically resistant layer or layers of the skin, the substance can be driven from the supply matrix through the needles directly to the targeted treatment site bypassing the high electrically resistant skin layer(s).

In another embodiment hereof, groups of the applicators may be provided, for example, on sheet material whereby the applicators are separable, e.g., by perforation lines through the sheet. Thus, the involved area of the applicator overlying the treatment site can be varied in size. A multi-channel electrode array is therefore coupled to the applicators whereby the area coverage of the applicators can be personalized to the size of the targeted treatment site. It will be appreciated that the shape of the applicators can vary, e.g., circular, rectilinear, hexagonal or any other shape. In this manner, the needles provide multiple very low electrically resistant pathways through the high electrically resistant layer(s) enabling, for example a micro-processor to drive via the multi-channel electrode array the medicament or a carrier therefore disposed in a matrix within the applicator through the skin to apply the medicament directly to the targeted treatment site.

In a preferred embodiment of the present invention, there is provided a device for delivering a medicament to a treatment site underlying an electrically resistant layer of an individual's skin, comprising an applicator for overlying the treatment site and the electrically resistant skin layer, the applicator having a plurality of needles projecting from a first surface thereof for penetrating the electrically resistant layer of the individual's skin, the needles and the surface being formed of a non-electrically conductive material; a matrix carried by the applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site; the applicator having a second surface formed of electrically conductive material.

In a further preferred embodiment, there is provided a system for delivering a medicament to a treatment site underlying an electrically resistant layer of an individual's skin, comprising an applicator for overlying the treatment site and the electrically resistant skin layer, the applicator having a plurality of needles projecting from one side thereof for penetrating the electrically resistant layer of the individual's skin; a matrix carried by the applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site; a first electrode for electrical connection with a power source; whereby, upon application of the applicator to the individual's skin overlying the treatment site and connection to the power source and a second electrode for electrical connection with the power source enabling completion of an electrical circuit through the first electrode, the medicament or the electrical carrier therefor, a portion of the individual's body, the second electrode and the power source, the system enables an electrical current to flow for electrokinetically driving the medicament or the medicament and the electrical carrier therefor through the needle orifices into the treatment site bypassing the electrically resistant layer of the individual's skin.

In a still further preferred embodiment, there is provided a system for delivering a medicament to a treatment site underlying an electrically resistant layer of an individual's skin, comprising a power source; an applicator for overlying the treatment site and the electrically resistant skin layer, the applicator having a plurality of needles projecting from one side thereof for penetrating the electrically resistant layer of the individual's skin; a matrix carried by said applicator for containing the medicament or the medicament and an electrical carrier therefor, the needles having one or more orifices in communication with the medicament or the medicament and the electrical carrier therefor contained in the matrix and opening at locations spaced from the matrix for delivering the medicament to the treatment site; a first electrode carried by the applicator in electrical connection with the power source; a second electrode in electrical connection with the power source; whereby, upon application of the applicator to the individual's skin overlying the treatment site and electrical connection to the power source and a second electrode for electrical connection with the power source enabling completion of an electrical circuit through the first electrode, the medicament or the electrical carrier therefor, a portion of the individual's body, the second electrode and the power source, the system enables an electrical current to flow to electrokinetically drive the medicament or the medicament and the electrical carrier therefor through the needle orifices into the treatment site bypassing the electrically resistant layer of the individual's skin.

Another preferred embodiment of the present invention includes a system for delivering a medicament to a treatment site underlying an electrically resistant layer of an individual's skin, comprising a sheet of discrete applicators selectively separable from one another enabling one or more of the applicators to overlie the treatment site and the electrically resistant skin layer, each applicator having a plurality of needles projecting from one side thereof for penetrating the electrically resistant layer of the individual's skin; a matrix carried by each applicator for containing the medicament or the medicament and an electrical carrier therefor, the needles of each applicator having one or more orifices in communication with the medicament or the medicament and the electrical carrier therefor contained in the matrix and opening at locations spaced from the matrix for delivering the medicament to the treatment site; a first electrode carried by each applicator for electrical connection with a power source; whereby, upon application of one or more of the applicators to the individual's skin overlying the treatment site and connection to the power source and a second electrode in electrical connection with the power source enabling completion of an electrical circuit through the first one or more electrodes, the medicament or the electrical carrier therefor of the one or more applicators, a portion of the individual's body, the second electrode and the power source, the system enables an electrical current to flow for electrokinetically driving the medicament or the medicament and the electrical carrier therefor through the needle orifices of the one or more applicators into the treatment site bypassing the electrically resistant layer of the individual's skin.

Still in a further embodiment, there is provided a method for delivering medicament to a treatment site underlying an electrically resistant layer of an individual's skin, comprising the steps of applying a plurality of micro-needles to the individual's skin to penetrate the electrically resistant layer of the individual's skin; and electrokinetically driving the medicament or the medicament and an electrical carrier therefore through the micro-needles into the treatment site bypassing the electrically resistant layer of the individual's skin.

Other certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; all of which are incorporated herein by reference in their entirety. Some or all of the above teaching therein may be applied to microneedle devices, their manufacture, and their use in iontophoretic applications.

The following Examples further illustrate the present invention but should not be construed as in any way limiting its scope.

### EXAMPLES

### Example 1

### In Vitro Iontophoretic Delivery of Tetracycline Through Hairless Rat Skin

Methodology:

In Vitro Skin Permeation Studies:

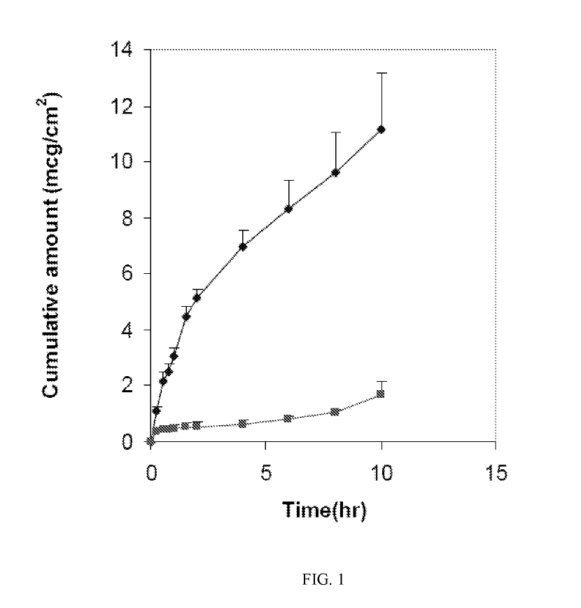

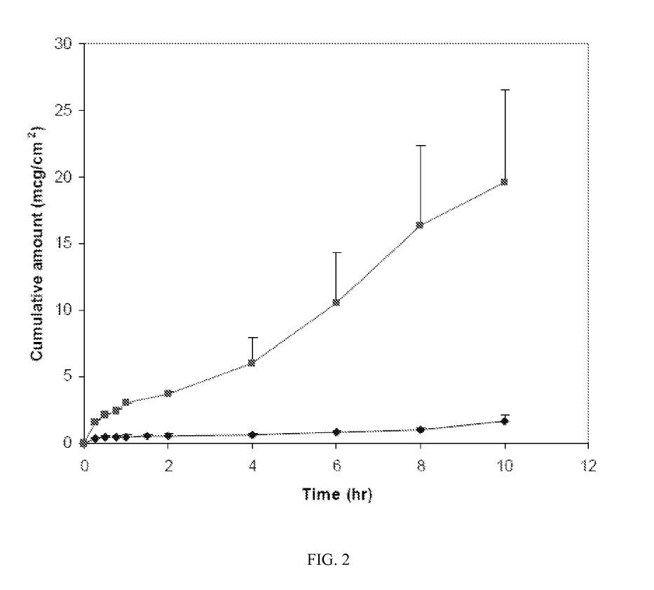

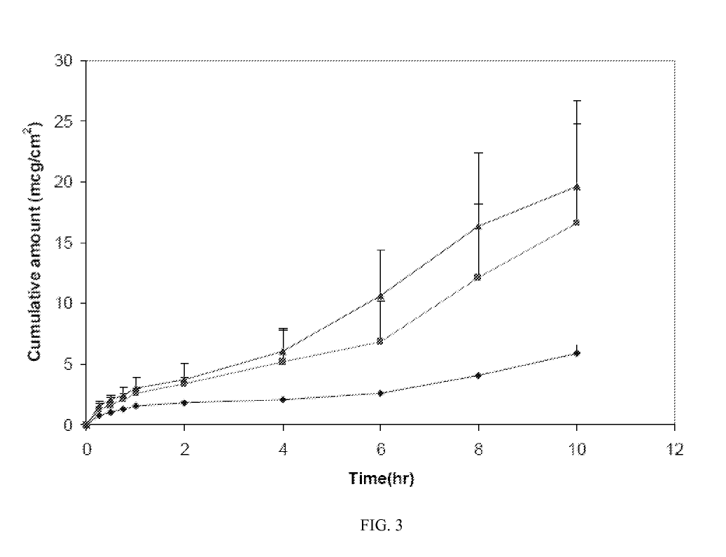

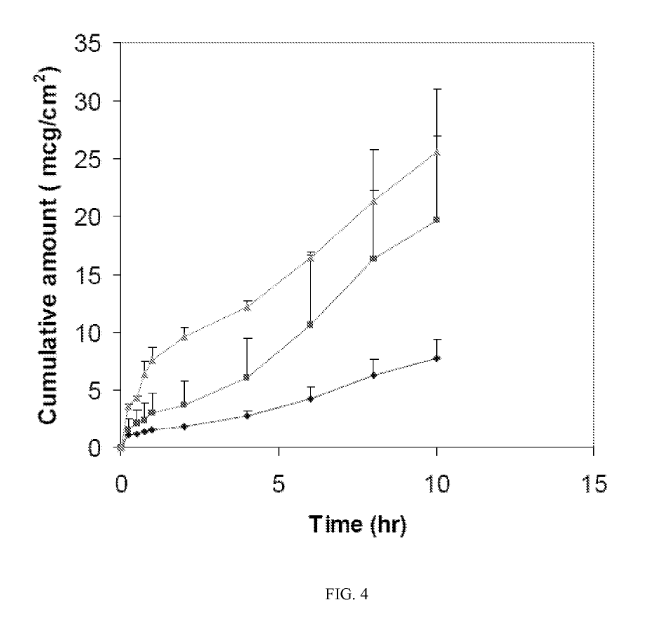

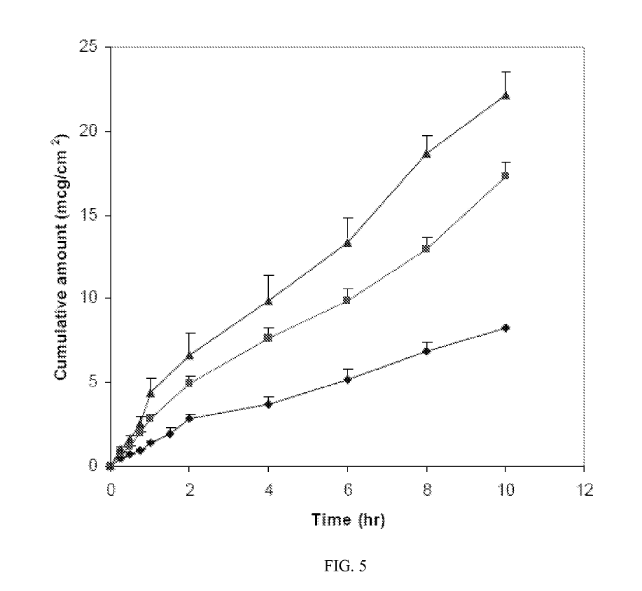

Freshly excised full thickness hairless rat skin (approximately 1 mm thick) was mounted in between the donor and receptor compartments of the horizontal diffusion cells with the stratum corneum facing the donor compartment.
The receptor compartment contained phosphate buffer (4 ml) at pH 7.4, and the donor compartment contained one of the following tetracycline hydrochloride solutions (4 ml): water (pH 2.1, 37 mg/ml), citrate buffer (pH 3.0, 9 mg/ml), phosphate buffer (pH 8.0, 0.7 mg/ml), or Teorell buffer (pH 8.3, 5.9 mg/ml, pH 11.9: 20 mg/ml).
The apparatus was maintained at 32° C., with constant stirring in both the donor and receptor compartments using magnetic stir bars to maintain sink conditions.
Silver/Silver chloride electrodes were used for iontophoresis. The area of skin exposed to the donor was 0.64 cm².
Current densities of 0.2 and 0.3 mA/cm² were applied for 1 h; and current density of 0.4 mA/cm² was applied for 30 min, 1 h and 2 h. Samples were collected for a period of 10 h. Both anodal and cathodal iontophoresis were performed.
At predetermined intervals, 0.5 ml of sample was collected from the receptor chamber and replenished with 0.5 ml of phosphate buffer (pH7.4).
   Analysis of Tetracycline Hydrochloride:
Samples from the receptor chamber were analyzed for the amount of tetracycline hydrochloride using a Shimadzu HPLC with a reverse phase column (Phenomenex, Luna C18 column with 5μ pore size).
The composition of the mobile phase used was 0.01N oxalic acid and acetonitrile (75:25) at a flow rate of 0.8 ml/min using a detection wavelength of 360 nm.
   Results:
The cumulative amount of drug permeating through the skin passively at the end of 10 h was 1.7±0.45 μg/cm² (FIG. 1).
Iontophoresis improved the delivery of the drug across the skin. i) The cumulative amount permeated through the skin at the end of 10 h by anodal iontophoresis for 1 h was 11.18±2.02 μg/cm². ii) The cumulative amount of the drug delivered at the end of 10 h by cathodal iontophoresis for 1 h was 19.65±6.9 μg/cm² (FIGS. 1 and 2). The current density used was 0.4 mA/cm² in both the cases.
The cumulative amount of drug permeated through the skin increased with an increase in the current density. The cumulative amount of the drug permeated through the skin over 10 hours increased from 5.85±0.72 μg/cm² to 19.65±6.9 μg/cm² with an increase in the current density from 0.2 mA/cm² to 0.4 mA/cm² (FIG. 3).
The amount of drug permeated through the skin increased with an increase in the duration of current application. The cumulative amounts of drug permeated through the skin over 10 hrs by iontophoresis with a current strength of 0.4 mA/cm² for 30 min, 1 h and 2 h were 7.76±1.67 μg/cm², 19.65±6.9 μg/cm² and 25.62±1.33 μg/cm² respectively (FIG. 4).
Dose response studies revealed enhanced delivery of the drug at higher donor concentrations. For a donor concentration of 10 mg/ml, 22.11 μg/cm² of tetracycline hydrochloride permeated through the skin as compared to 8.25 μg/cm² delivered from a donor concentration of 2.5 mg/ml (FIG. 5).
   For all the above studies except anodal iontophoresis, tetracycline hydrochloride solution in borate buffer at pH 9.4 was employed. For anodal iontophoresis, tetracycline hydrochloride in citrate buffer at pH 3.0 has been used.

Tetracycline hydrochloride exists as an anion above pH 7.7, so cathodal iontophoresis at pH 9.4 has significantly increased the cumulative amount of drug permeated through the skin when compared to anodal iontophoresis or passive delivery. Cathodal iontophoresis showed significant enhancement of drug transport through skin as compared to anodal iontophoresis, which suggests that electrorepulsion is the major mechanism of drug transport through the skin and the contribution of electroosmosis is negligible.

The cumulative amount of drug permeated through skin was the greatest with cathodal iontophoresis at pH 9.2 followed by anodal iontophoresis at pH 3.0 and passive delivery. The cumulative amount of drug permeated through the skin increased with an increase in the donor concentration.

### Example 2

### In Vivo Topical Delivery of Tetracycline Using Intracutaneous Microanalysis

In vivo microdialysis studies were undertaken to determine the effect of current and time on the transdermal delivery of tetracycline using iontophoresis. The study placed a tetracycline formulation (10 mg/mL, pH ˜9.3) in a cartridge designed for iontophoresis. The cathode from a constant current source was connected to the cartridge and the anode was connected to a Trans Q (IOMED, Inc.) inactive electrode. Cathodal iontophoresis current was varied from 200-400 μA/cm² for a period of 0.5-4 hours. The microdialysis dialysate samples collected were analyzed using HPLC. Potential skin irritation was monitored using chromameter, laser Doppler velocimetry (LDV) and transepidermal water loss (TEWL).

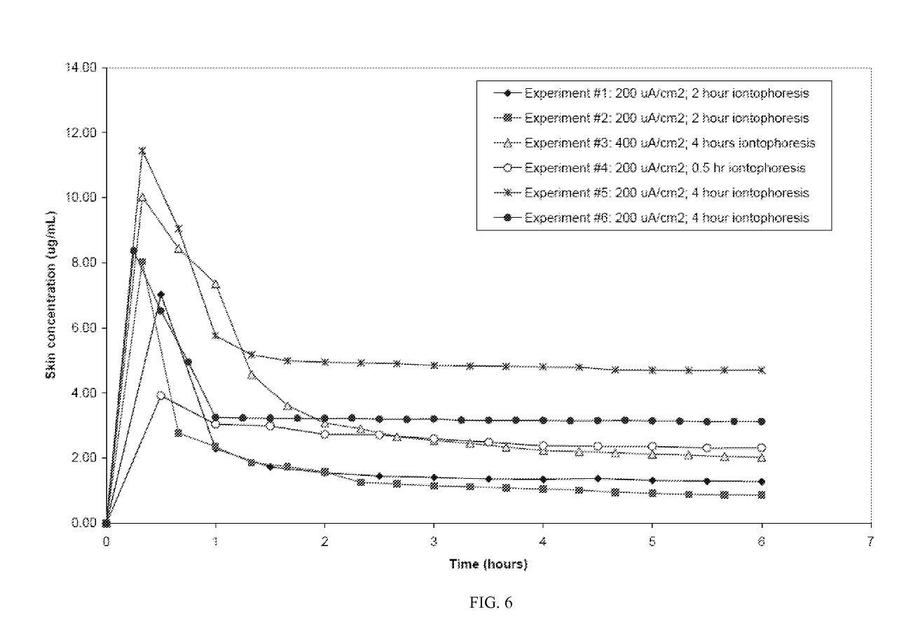

According to FIG. 6, the cumulative amount of drug permeated through the skin increased with an increase in the duration of current application. Thus, the highest amount of tetracycline concentration was found in the hairless rat skin with 4 hours of application time.

As used in this specification and the appended claims, the singular forms “a”, “an”, and “the” include plural referents unless the context clearly dictates otherwise.

While this invention has been particularly shown and described with references to preferred embodiments thereof, it will be understood by those skilled in the art that various changes in form and details may be made therein without departing from the scope of the invention encompassed by the appended claims.

1. A formulation suitable for iontophoresis comprising at least one tetracycline antibiotic at an ionic strength of at least about 0.05M.

2. The formulation of claim 1 wherein the tetracycline antibiotic is selected from tetracycline, chlortetracycline, oxytetracycline, demecloycline, doxycycline, lymecyline, meclocyline, methacycline, minocyline, rolitetracycline and tigecycline.

3. The formulation of claim 1 wherein the tetracycline antibiotic is selected from tetracycline and doxycycline.

4. The formulation of claim 1 wherein the formulation further comprises a viscosity modulating agent selected from cellulosic polymers and derivatives thereof, crosslinked acrylic acid polymers, hydrophilic polymers, gums, sodium alginate; gelatin and any combination thereof.

5. A formulation suitable for iontophoresis comprising tetracycline at an ionic strength of at least about 0.1M.

6. The formulation of claim 5 wherein the ionic strength is at least about 0.25M.

7. The formulation of claim 6 wherein the iontophoresis is cathodal and performed at a pH of at least about 7.5.

8. The formulation of claim 7 wherein the pH is about 9.

9. The formulation of claim 6 wherein the iontophoresis is anodal and performed at a pH of less than about 4.

10. The formulation of claim 9 wherein the formulation comprises a viscosity modulating agent selected from cellulosic polymers and derivatives thereof, crosslinked acrylic acid polymers, hydrophilic polymers, gums, sodium alginate; gelatin and any combination thereof.

11. A formulation suitable for cathodal iontophoresis comprising tetracycline at a pH of at least about 7.5.

12. The formulation of claim 11 wherein the pH is about 9.

13. The formulation of claim 12 wherein the formulation comprises a viscosity modulating agent selected from cellulosic polymers and derivatives thereof, crosslinked acrylic acid polymers, hydrophilic polymers, gums, alginate; gelatin and any combination thereof.

14. A formulation suitable for anodal iontophoresis comprising tetracycline at a pH of less than about 4.

15. The formulation of claim 14 wherein the formulation comprises a viscosity modulating agent selected from cellulosic polymers and derivatives thereof, crosslinked acrylic acid polymers, hydrophilic polymers, gums, alginate; gelatin and any combination thereof.

16. A method for administering tetracycline to a patient comprising iontophoretically administering to the body surface of a patient in need thereof, the formulation of claim 1.

17. The method of claim 16 wherein a current density of at least about 0.02 mA/cm² is applied.

18. The method of claim 17 wherein the current density is at least 0.1 mA/cm².

19. The method of claim 16 wherein the tetracycline is administered at a flux of at least about 5 μg/cm² per hour.

20. The method of claim 19 wherein the flux is at least about 10 μg/cm² per hour.

21. An iontophoretic device having micro-needles formed on a contacting surface comprising a formulation according to claim 1.

22. A method for delivering tetracycline antibiotic to a treatment site underlying the skin of an individual, the method comprising: 1) applying a plurality of micro-needles to the skin to penetrate the skin; and electrokinetically driving the tetracycline antibiotic at an ionic strength of at least about 0.05M and an electrical carrier therefore through pores in the skin formed by the micro-needles and into the treatment site.

23. A method of claim 22 wherein the tetracycline antibiotic is selected from tetracycline, chlortetracycline, oxytetracycline, demecloycline, doxycycline, lymecyline, meclocyline, methacycline, minocyline, rolitetracycline and tigecycline.

24. The method of treating acne comprising iontophoretically administering to the body surface of a patient in need thereof, the formulation of claim 1.

25. A formulation of claim 1 free of dermal penetration enhancer which is selected from the group consisting of 1-alkylazacyloheptan-2-one, said alkyl having from 8 to 16 carbon atoms, and a cis-olefin of the formula CH₃(CH₂)ₓCH═CH(CH₂)_{y}R³ where R³ is CH₂OH, CH₂NH₂ or COR⁴, and R₄ is OH or (C₁-C₄)alkoxy, x and y are each an integer from 3-13 and the sum of x and y is from 10 to 16.
